(19) United States
(12) Patent Application Publication
(10) Pub. No.: US 2016/0067031 A1
(43) Pub. Date: Mar. 10, 2016

(54) METHODS AND USES OF MEDIASTINAL PLEURA TISSUE FOR VARIOUS STENT AND OTHER MEDICAL APPLICATIONS

(71) Applicants: Ghassan S. Kassab, La Jolla, CA (US); Xiao Lu, San Diego, CA (US)

(72) Inventors: Ghassan S. Kassab, La Jolla, CA (US); Xiao Lu, San Diego, CA (US)

(21) Appl. No.: 14/847,695

(22) Filed: Sep. 8, 2015

Related U.S. Application Data
(60) Provisional application No. 62/047,206, filed on Sep. 8, 2014.

(51) Int. Cl. A61F 2/06; A61F 2/82; A61L 27/36; A61K 35/34; A61L 27/54; A61L 27/58; A61F 2/24; A61F 2/07

(52) U.S. Cl. CPC A61F 2/062; A61F 2/06; A61F 2/2415; A61F 2/82; A61F 2/07; A61K 35/34; A61L 27/54; A61L 27/58; A61L 27/3691; A61L 27/3604; A61L 27/3683

(57) ABSTRACT

Methods, products and uses of or relating to biological tissues for various stent and other medical applications are disclosed. In an exemplary embodiment of a method of processing a tissue of the present disclosure, the method comprises the steps of acquiring a tissue comprising at least a portion of parietal pleura, selecting a sample of mediastinal pleura tissue from the parietal pleura tissue, and fixing the sample of mediastinal pleura tissue using a fixative, resulting in a fixed sample. Furthermore, in an embodiment of a tissue product of the present disclosure, the tissue product comprises a frame configured to retain mediastinal pleura tissue thereon and such tissue coupled to the frame.

### PRIORITY

The present application is related to, and claims the priority benefit of, U.S. Provisional Patent Application Ser. No. 62/047,206, filed Sep. 8, 2014, the contents of which are hereby incorporated by reference in their entirety into the present disclosure.

### BACKGROUND

To date, the small intestinal submucosa (SIS) is the major biological tissue scaffold that has garnered some biological applications to replace or augment injured or damaged biological tissues. Once the smooth muscles are stripped away, the SIS consists of largely collagen and some elastin fibers. The fixation of the tissue, however, renders the scaffold stiff and can result in losses of some of its biological advantages.

In view of the same, it would be advantageous to identify and process an effective alternative tissue that would maintain its elasticity, keep its biological advantages, and be useful for various bodily purposes, including as part of various medical devices.

### BRIEF SUMMARY

In an exemplary embodiment of a method of processing a tissue of the present disclosure, the method comprises the steps of acquiring a tissue comprising at least a portion of parietal pleura tissue from a mammal, selecting a sample of mediastinal pleura tissue from the parietal pleura tissue, and fixing the sample of mediastinal pleura tissue using a fixative, resulting in a fixed sample. In an exemplary embodiment of a method of processing a tissue of the present disclosure, the method may further comprise the step of placing the sample of mediastinal pleura tissue within or upon a mount having known dimensions. Additionally or alternatively, embodiments of the method may also comprise the step of forming the fixed sample of mediastinal pleura tissue into a product. In such embodiment of the method, the product may be selected from the group consisting of a stent cover, a diaphragm cover, a hernia repair cover, a brain cover, a general organ cover, a wound cover, a prosthetic device cover, a skull cover, a general tissue cover, a valve, a patch, a surgical membrane, a skin substitute, a suture reinforcement, a tubular structure, a tendon replacement, a bladder tissue replacement, a urethra tissue replacement, a vaginal tissue replacement, a muscle replacement or other tissue replacement products. Furthermore, the product may comprise a first surface and a second surface, and the composition of the product may include elastin fibers. In such embodiments, the first and second surfaces of the product are lined with mesothelial cells.

In at least one embodiment of the method of the present disclosure, the step of acquiring a tissue comprising at least a portion of parietal pleura tissue from a mammal may comprise acquiring the parietal pleura by way of dissecting or resecting tissue from a deceased mammal. Additionally or alternatively, the step of acquiring a tissue comprising at least a portion of parietal pleura tissue from a mammal may comprise acquiring the at least a portion of parietal pleura from a mammal selected from the group consisting of a pig, a horse, a cow, a goat, a sheep, and a human.

In yet another embodiment, the step of selecting a sample of mediastinal pleura tissue further comprises selecting a sample of mediastinal tissue from a section of the parietal pleura tissue positioned between a left lung and a right lung of the mammal. The step of selecting a sample of mediastinal pleura tissue may alternatively or additionally comprise isolating and harvesting a sample of mediastinal pleura tissue from a section of the parietal pleura tissue that extends between a pericardium and a diaphragm of the mammal. Furthermore, in at least one embodiment of a method of processing a tissue of the present disclosure, the step of selecting a sample of mediastinal tissue from the parietal pleura tissue further comprises the step of removing lymph nodes and fatty material from the portion of parietal pleura tissue.

In an exemplary embodiment of a method of processing a tissue of the present disclosure, the method may further comprise the steps of positioning the fixed sample of mediastinal pleura tissue upon at least a portion of a frame, wherein the fixed sample of mediastinal pleura tissue and the frame collectively form a tissue product, and positioning the tissue product within a mammalian lumen so that fluid native to the mammalian lumen may pass through a lumen defined within the tissue product. In an exemplary embodiment, the fixed sample of mediastinal pleura tissue comprises tissue having stretchability and durability properties sufficient to allow the fixed sample of mediastinal pleura tissue to move relative to the fluid flow through the lumen defined within the tissue product. Furthermore, in at least one embodiment, the tissue product comprises a valve having a leaflet configuration. Such valve may have, for example, a bileaflet configuration or a trileaflet configuration.

Other embodiments of the method of processing a tissue of the present disclosure further comprise the step of decellularizing at least a portion of the sample of the mediastinal pleura tissue prior to performing the fixing step. Additionally or alternatively, such methods may also comprise the step of treating a patient using the fixed sample of mediastinal pleura tissue.

In an exemplary embodiment of a tissue product according to the present disclosure, the tissue product comprises a frame configured to retain a mammalian tissue thereon and the mammalian tissue coupled to the frame. In such embodiments, the mammalian tissue comprises mediastinal pleura tissue and the product is configured such that when the product is positioned within a mammalian lumen, fluid native to the mammalian lumen may pass through a lumen defined within the product. Furthermore, in at least one embodiment, the product comprises an outer surface and an intraluminal surface and the composition of the product includes elastin fibers. Additionally, in such embodiments, the outer and intraluminal surfaces of the product may be lined with mesothelial cells. In yet another embodiment, the product is configured for mammalian treatment or therapy.

In an additional exemplary embodiment of a method of the present disclosure, the method comprises the steps of shaping a mammalian mediastinal pleura tissue so that the mammalian mediastinal pleura tissue fits around at least a portion of a frame, positioning the mammalian mediastinal pleura tissue around a mount, positioning at least a part of the frame around the mammalian mediastinal pleura tissue positioned around the mount, and connecting the mammalian mediastinal pleura tissue to the frame to form a tissue product. In yet an additional embodiment, the tissue product of the aforementioned method is configured for mammalian treatment or therapy.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a 60× microscopic view of a portion of swine mediastinal pleura according to an exemplary embodiment of the present disclosure;

FIGS. 2A and 2B show a swine pulmonary ligament connected to a lung, according to exemplary embodiments of the present disclosure;

FIG. 3A shows a close-up view of a swine pulmonary ligament, according to an exemplary embodiment of the present disclosure;

FIG. 3B shows a portion of a pulmonary ligament held in place upon a frame, according to an exemplary embodiment of the present disclosure;

FIGS. 4A and 4B show a fixed product, according to exemplary embodiments of the present disclosure;

FIGS. 5A-5D show various depictions of a portion of a pulmonary ligament, after fixation, formed into an exemplary constructed valve according to exemplary embodiments of the present disclosure;

FIGS. 6A-6D show various processed mammalian tissue products in various configurations, according to exemplary embodiments of the present disclosure;

FIG. 6E shows a block diagram of components of a kit, according to an exemplary embodiment of the present disclosure;

FIGS. 6F and 6G show mammalian tissue and tissue harvest locations, according to exemplary embodiments of the present disclosure;

FIGS. 7A and 7B show a bileaflet frame configuration, according to an exemplary embodiment of the present disclosure;

FIG. 8A shows a portion of a mammalian tissue cut/shaped to fit along a bileaflet frame, according to an exemplary embodiment of the present disclosure;

FIGS. 8B and 9A show how portions of mammalian tissue can be positioned within/around portions of a frame, according to exemplary embodiments of the present disclosure;

FIG. 9B shows an exemplary product having a bileaflet frame and a tissue positioned thereon, according to an exemplary embodiment of the present disclosure;

FIGS. 10A and 10B show a trileaflet frame configuration, according to an exemplary embodiment of the present disclosure;

FIG. 11A shows a portion of a mammalian tissue cut/shaped to fit along a trileaflet frame, according to an exemplary embodiment of the present disclosure;

FIG. 11B shows an exemplary product having a trileaflet frame and a tissue positioned thereon, according to an exemplary embodiment of the present disclosure;

FIG. 11C shows an exemplary product configured as a valve and positioned within a mammalian luminal organ, according to an exemplary embodiment of the present disclosure; and

FIG. 12 shows steps of a method to manufacture a product, according to an exemplary embodiment of the present disclosure.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure contains disclosure of novel methods and uses for harvesting and applying certain mammalian tissue for use in connection with various medical applications. The mammalian pulmonary ligament, the mammalian visceral pleura, and the mammalian mediastinal pleura, as referenced in detail below and disclosed within the present application, can be harvested, fixed, and used for a number of medical applications previously unknown and not identified in the medical arts. In at least one embodiment of the present disclosure, mediastinal pleura tissue is identified, harvested, fixed, and ultimately used in connection with mammalian treatment/therapy. As referenced herein, pulmonary ligament and visceral pleura are both “pulmonary region” tissue. Alternatively, mediastinal pleura is parietal pleura, which is the portion of the pleura external to the pulmonary pleura.

As referenced in detail herein, it is advantageous to identify and process thin scaffold biological tissue that consists of largely elastin and some collagen fibers (the converse of SIS), since elastin is not as prone to fixation as collagen fibers. Hence, fixation of tissue with elastin largely maintains its elasticity and hence biological mechanical activity. Furthermore, it is advantageous to identify a thin membranous native tissue that does not require any processing, such as stripping of muscle or treatment with antibiotics given the bacteria flora such as present within the intestines. Finally, there is significant advantage to tissue that has epithelial layers (e.g., mesothelial cells) on both sides of the tissue as such layers tend to provide a slippery, non-adhesive and protective surface. As referenced in detail herein, the present disclosure includes uses and methods in connection with such a biological tissue and processing steps for various biological applications.

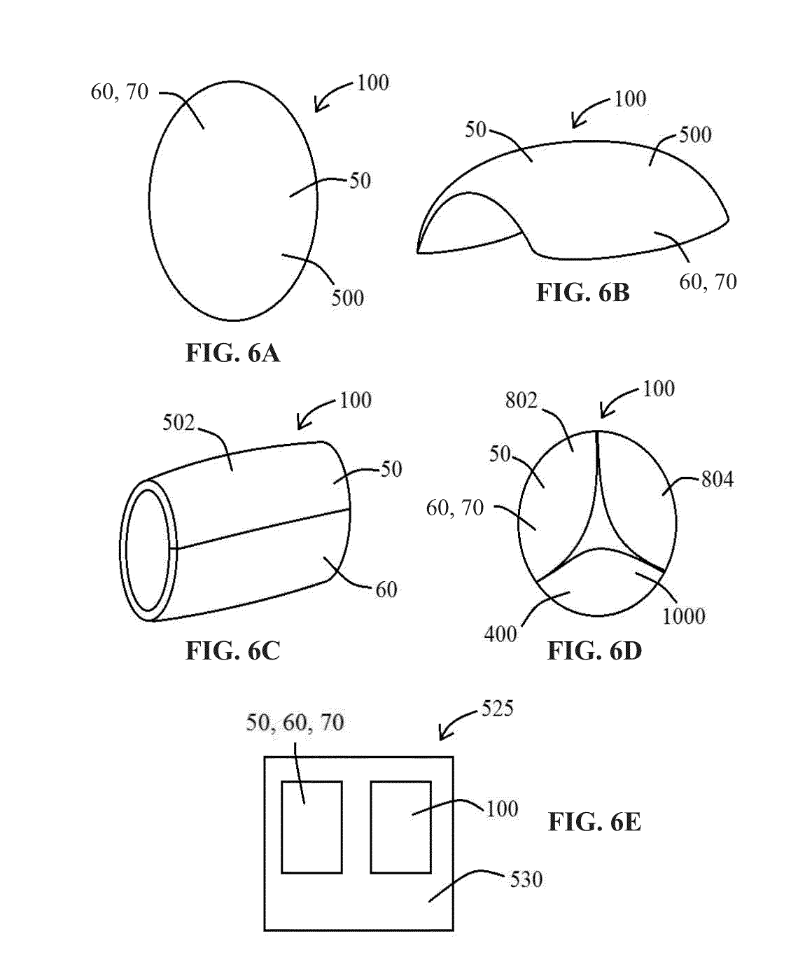

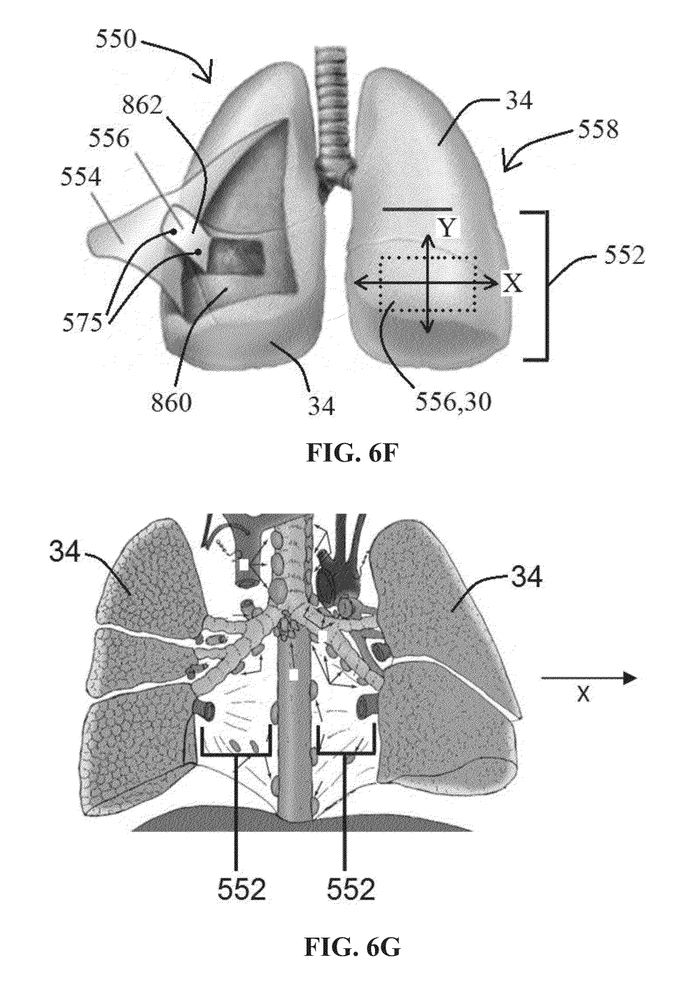

A pleura is a serous membrane that folds back onto itself to form a two-layered membrane structure. Generally, the outer pleura (parietal pleura 554) lines the thoracic cavity, whereas the inner pleura (visceral or pulmonary pleura) covers the lungs. The parietal pleura 554 lines the inner surface of the chest wall, covers the superior surface of the diaphragm and encases all of the thoracic viscera (excluding the lungs) (see FIG. 6F). Accordingly, the parietal pleura 554 separates the pleural cavity (where the lungs are positioned) from the mediastinum or the “middle” section of the chest cavity.

The parietal pleura 554 is divided into different portions according to its position. For example, the costal pleura is the portion of the parietal pleura 554 that lines the inner surfaces of the ribs and intercostals, the diaphragmatic pleura is that which lines the convex surface of the diaphragm, and the cervical pleura is the portion that rises into the neck and over the apex of the lung. Furthermore, mediastinal pleura 38 is the portion of parietal pleura 554 that defines the mediastinum and encases all of the thoracic viscera except for the lungs, as it runs therebetween.

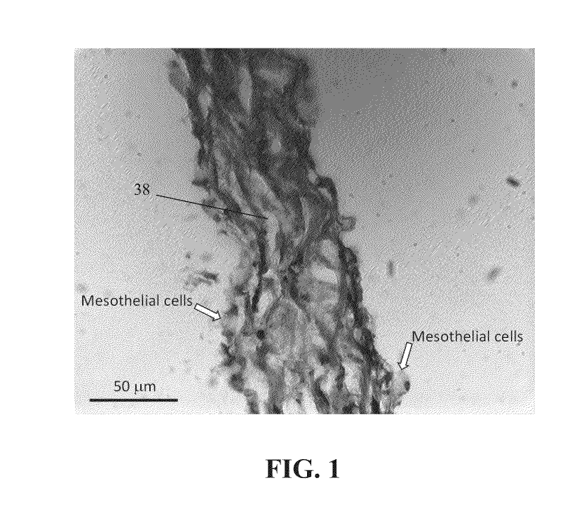

As the mediastinal pleura 38 separates the right and left lungs, inflation of the lungs causes a corresponding expansion of the mediastinal membrane, thereby resulting in significant friction between the mediastinal pleura 38 and the lungs' surfaces during breathing. While the mediastinal pleura 38 is relatively thin, it nevertheless exhibits substantial integrity and elasticity to accommodate the lungs' expansion and tolerate the friction imposed thereby. The significant elasticity of the mediastinal tissue is indicative of its composition, which consists of multiple fiber-sheet layers having an abundance of elastin fibers in addition to the collagen typically present in connective tissue. This is significant from a tissue product and medical application perspective as, unlike collagen, elastin cannot be fixed and largely retains its elasticity and biomechanical activity post-fixation. Furthermore, both surfaces of the mediastinal pleura 38 are lined with mesothelium, which provides for significant integrity, promotes its friction-resistant nature, and provides for antithrombotic properties. For example, FIG. 1 shows a microscopic view of a cross-section of swine mediastinal pleura 38 having an average thickness of about 70 microns and having its two surfaces lined in mesothelial cells. Accordingly, post fixation mediastinal pleura 38 (which could also be referred to as processed mediastinal pleura 70 of the present disclosure and potentially configured as an exemplary product 100 of the present disclosure as referenced below) has high elasticity, and both sides of the mediastinal pleura 38 tissue are smooth and covered with an epithelial layer that secrets a lubricant. Due at least to these advantageous properties, and as described otherwise herein, the novel nature of identifying, harvesting, fixing and using the processed mediastinal pleura 70 tissue can result in numerous therapies and treatments not previously considered or used in the medical arts.

The visceral pleura covers the lungs and extends to the hilum where it becomes continuous with the parietal pleura. As the anterior and posterior pleural extend below the pulmonary veins, the two layers of pleura come together to form the inferior pulmonary ligament. Hence, the pulmonary ligament is a double layer of pleura that drapes caudally from the lung root and loosely tethers the medial aspect of the lower lobe of the lung to the mediastinum. However, and importantly, the pulmonary ligament does not functionally behave the same as two layers of pleura, as the non-isotropy of pulmonary ligament tissue is notably different than just two layers of pleura. Furthermore, the degree of collagen within pulmonary ligament is also different than in two layers of pleura, and the function of pulmonary ligament is also different, as pulmonary ligament tissue resists load in one direction. The pulmonary ligament tethers the lung and has substantial elasticity (over 200% extension, which may be a lateral extension) to expand with each inflation of the lung. Similar to the mediastinal pleura 38 previously discussed, the significant elasticity of the pulmonary ligament tissue stems from its high elastin content, which is beneficial in that it largely retains its elasticity post fixation.

In certain embodiments of processed pulmonary ligament 50, processed visceral pleura 60, and/or processed mediastinal pleura 70 of the present disclosure, said tissues can have a microarchitecture including non-randomly oriented collagen and elastin fibers, which can be retained from the native microarchitecture of the processed pulmonary ligament 50, processed visceral pleura 60 and/or processed mediastinal pleura 70, and/or the processed pulmonary ligament 50, processed visceral pleura 60 and/or processed mediastinal pleura 70 can exhibit an anisotropic elastic character, for example as can be demonstrated in biaxial stretch testing and/or through optical and/or microscopic visualization of the tissue microstructure. As well, in these and other embodiments, processed pulmonary ligament 50 tissue can have a thickness of about 80 microns to about 100 or 120 microns, and even as high as about 300 microns, including thicknesses between about 90 microns and 100 microns, which depends upon the species from which the pulmonary ligament tissue is obtained. Processed visceral pleura 60 may have a smaller thickness, such as between about 40 microns and about 80 microns, as referenced further herein. Furthermore, processed mediastinal pleura 70 may have a thickness of about 50 to about 80 microns. Other embodiments of processed pulmonary ligament 50, processed visceral pleura 60, and/or processed mediastinal pleura 70 of the present disclosure may be up to 300 microns in thickness in some regions. In an actual exemplary sample of 15 harvested processed pulmonary ligament samples obtained according to the present disclosure, the average thickness was 102 microns, and the thickness range was from 22 microns to 269 microns. Different thicknesses of tissue may be preferred for different embodiments, such as relatively thinner tissues for valve applications, and relatively thicker tissues for hernia repair, for example.

For various pulmonary ligament 30 and/or visceral pleura 556 samples, a predominant proportion of the collagen fibers in the tissue are oriented generally in a first direction, with that direction extending substantially parallel to the median (or midsagittal) plane of the animal from which the tissue was harvested. For example, and in at least one embodiment, at least 75% of collagen fibers within the harvested pulmonary ligament 30 and/or visceral pleura 556 tissue are oriented in a first direction. In at least another embodiment, at least 60% of collagen fibers within the harvested pulmonary ligament 30 and/or visceral pleura 556 tissue are oriented in a first direction. Furthermore, and in various pulmonary ligament 30 and/or visceral pleura 556 samples, said tissues include elastin fibers that extend in a direction transverse to that of the predominating collagen fibers contained therein. However, with respect to mediastinal pleura, collagen fibers do not orient in any particular direction and, hence, the mediastinal pleura 38 is mechanically more isotropic than the pulmonary ligament 30 and/or visceral pleura 556.

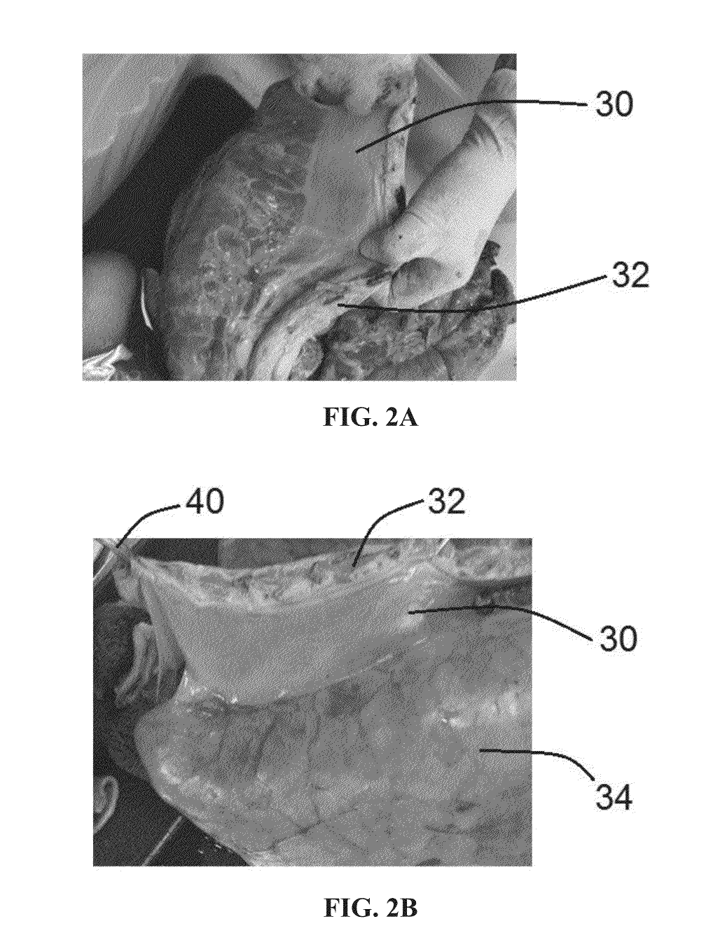

FIGS. 2A and 2B pictorially show a swine pulmonary ligament (an exemplary ligament 30) by way of gripping a portion of the pig (such as by the aorta and/or esophagus (collectively shown as 32 in FIG. 2A) or tissue in that general vicinity) and pulling the same away from the lung 34, as shown in FIG. 2B. Gripping and/or separation of tissue can be performed by hand, as shown in FIG. 2A, and/or by using forceps 40, as shown in FIG. 2B. The pulmonary ligament 30 is clearly shown and identified in FIGS. 2A and 2B.

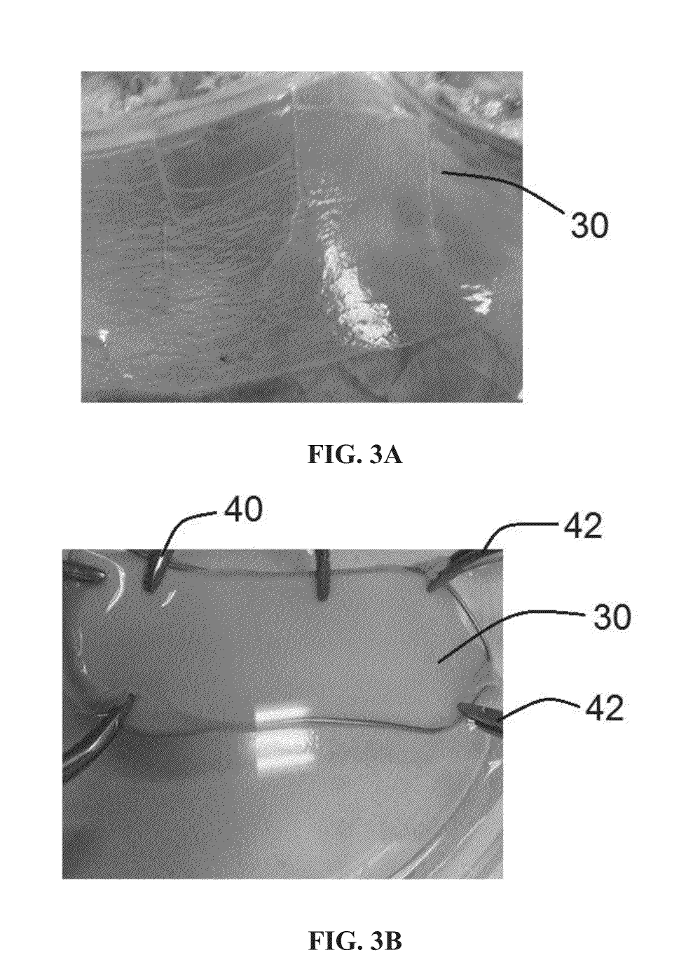

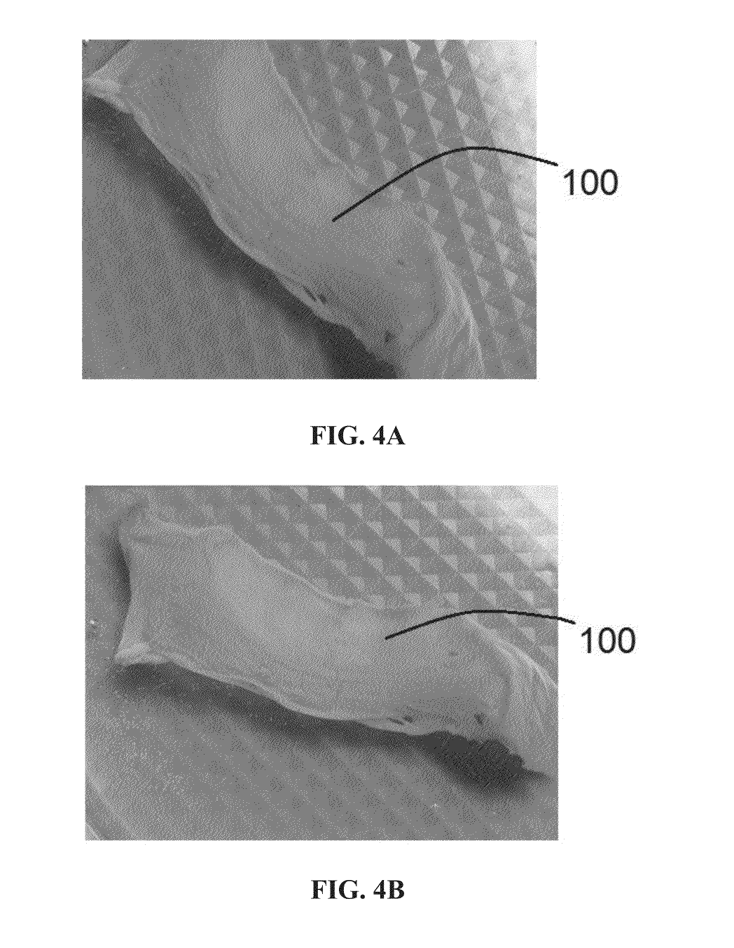

FIG. 3A shows a closer view of a portion of the pulmonary ligament 30, and FIG. 3B shows a portion of the pulmonary ligament 30 held in place using a series of clamps 42 positioned around a mount 44, for example, and being fixed with a fixative, such as glutaraldehyde. Post fixation pulmonary ligament (which could also be referred to as a processed ligament 50 of the present disclosure, potentially configured as an exemplary product 100 of the present disclosure as referenced below), as shown in FIGS. 4A and 4B, has high elasticity, and both sides of the ligament tissue are smooth and covered with an epithelial layer that secretes a lubricant.

It will be appreciated that mediastinal pleura may be mounted and fixed in the same manner as described herein with respect to the pulmonary ligament 30 to result in processed mediastinal pleura 70. Additionally, the product 100 manufacturing processes and techniques described herein are not limited to the specific type(s) of tissue referenced in connection therewith. Indeed, while one type of tissue—porcine pulmonary ligament 30 for example—may be used to describe the manufacture and various aspects of a particular product 100, such tissue is included only for explanatory purposes and therefore is not limiting with respect to different embodiments of the product 100 (unless expressly stated otherwise). Instead, it will be appreciated that processed mediastinal pleura 70 may be used to form products 100 pursuant to the processes and methods set forth in the present disclosure, either alone or in combination with other types of biological tissue.

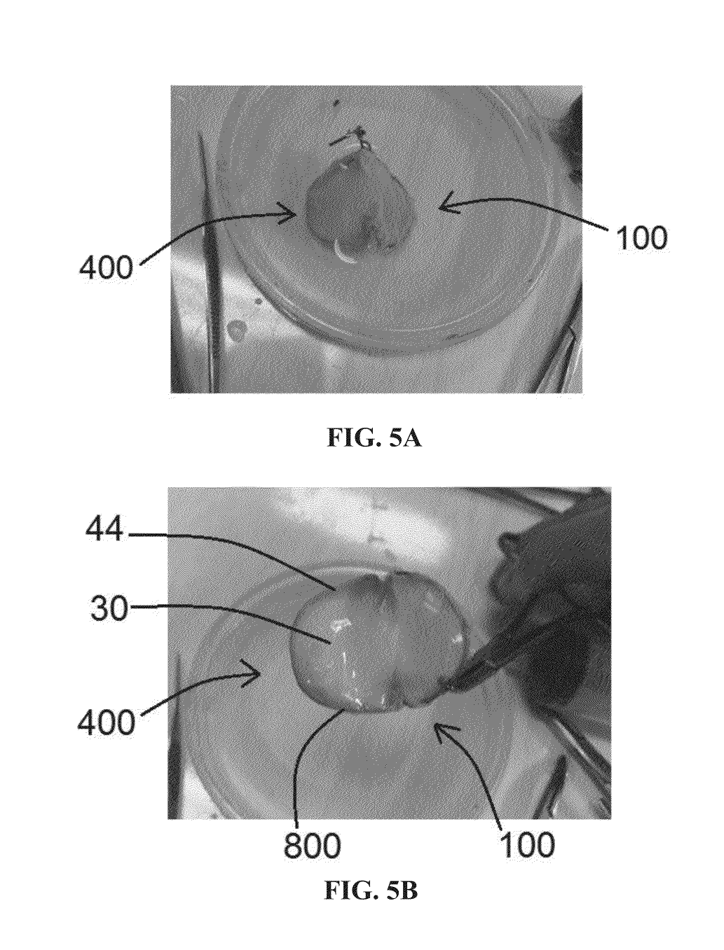

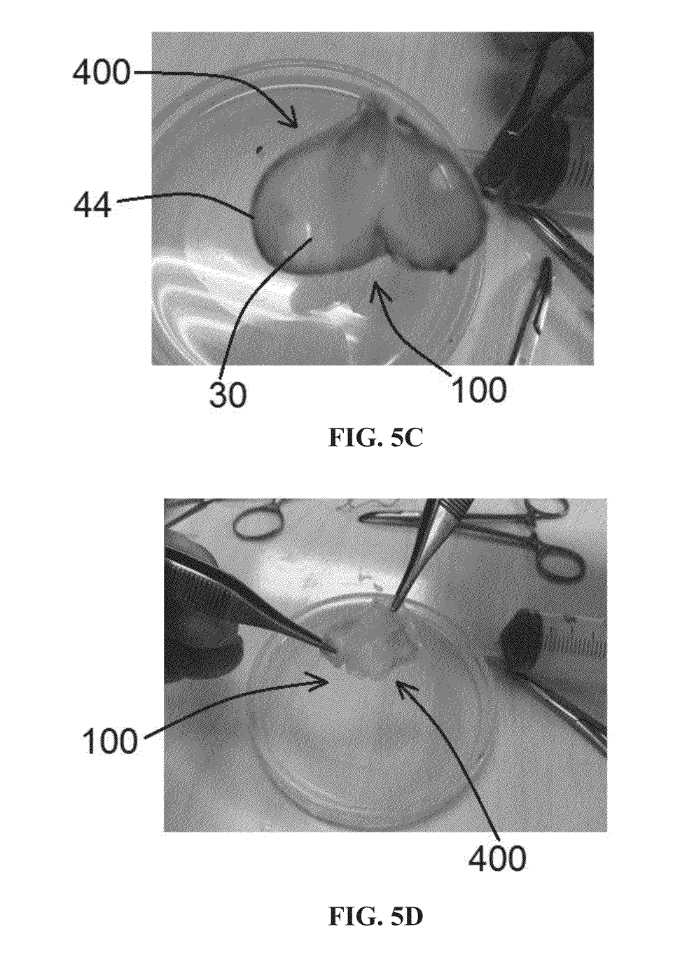

FIGS. 5A-5D show various depictions of a portion of a porcine pulmonary ligament 30, after glutaraldehyde fixation (to form processed ligament 50 and potentially an exemplary product 100), and formed into an exemplary constructed valve 400 of the present disclosure. For example, and as shown in FIGS. 3B and 5A-5C, pulmonary ligament 30 can be placed upon a mount 44, using one or more forceps 40 and/or clamps 42 (as shown in FIG. 3B), and/or one or more sutures 800 (as shown in FIG. 5B in connection with use of a mount 44, and as described in further detail herein in connection with one or more frames 600 of the present disclosure). Placement may also include folding portions of pulmonary ligament 30 around portions of mount 44, as shown by way of folded portion 48 in FIG. 5C. If one or more sutures 800 are used, said sutures 800 could comprise nylon or another suitable material, and could be placed using a needle (not shown), as described in further detail herein.

As shown in the various figures, the valve 400 (comprising pulmonary ligament in the embodiment shown), which is an exemplary processed product 100 of the present disclosure, easily flexes and maintains its shape. Products 100 can include processed ligament 50, processed visceral pleura 60 and/or processed mediastinal pleura 70, as referenced in further detail herein, and may also be referred to herein as medical articles of manufacture. As referenced herein, pulmonary ligament 30 refers to pulmonary ligament tissue that has not yet been processed, and processed ligament 50, optionally configured as one or more processed products 100 of the present disclosure, refers to tissue that has been processed, such as by fixation, and optionally configured as products 100. Similarly, and as also referenced herein, visceral pleura 556 refers to visceral pleura tissue that has not yet been processed, and processed visceral pleura 60, optionally configured as one or more processed products 100 of the present disclosure, refers to tissue that has been processed, such as by fixation, and optionally configured as products 100. Likewise, and as still further referenced herein, mediastinal pleura 38 refers to mediastinal pleura tissue that has not yet been processed, and processed mediastinal pleura 70 refers to tissue that has been processed, such as by fixation, and optionally configured as products 100. Various valves 400 of the present disclosure may comprises any number of valves, including, but not limited to, aortic valves, mitral valves, pulmonary valves, tricuspid valves, and/or other percutaneous valves.

FIGS. 6A-6D show various processed products 100 in various configurations, according to exemplary embodiments of the present disclosure. For example, FIG. 6A shows an exemplary product 100 of the present disclosure configured as a patch, membrane, tissue replacement, cover, or reinforcement. Said embodiments (patch, membrane, tissue replacement, cover, or reinforcement) shall be referred to generally as patches 500, as labeled in FIG. 6A. FIG. 6B shows another exemplary product 100 of the present disclosure configured as a curved patch, membrane, tissue replacement, cover, or reinforcement (collectively curved patches 500). FIG. 6C shows an exemplary product 100 configured as a tube 502, and FIG. 6D shows an exemplary product 100 configured as a valve 400. Valve 400, as shown in FIG. 6D, is configured as a tri-leaflet valve 400 (including, for example, leaflets 802, 804, and 1000, as referenced in further detail herein), but other valve 400 embodiments of the present disclosure may be single leaflet valves 400, bileaflet valves 400, or valves 400 with more than three leaflets. In each embodiment shown therein, products 100 comprise one or more processed ligaments 50, one or more processed visceral pleura 60 and/or one or more processed mediastinal pleura 70. However, it will be noted that processed mediastinal pleura 70 tissue is particularly suitable for use in connection with the manufacture of valve leaflets as mediastinal pleura 38 tissue is thin and relatively abundant with respect to the surface area that is available for harvesting and producing leaflets therefrom.

Other product 100 configurations are contemplated by the present disclosure, as various biological uses of products 100 can be had, and the present disclosure is not limited to the configurations shown in the figures. For example, a venous stent cover (an exemplary cover 502) with a membrane and valve 400 included is an exemplary product 100 of the present disclosure, with features shown in one or more figures referenced herein, such as FIGS. 6B, 6C, and 6D. For example, a product 100 of the present disclosure could have an external shape shown in FIG. 6C and a valve 400 as shown in FIG. 6D.

As for preparation of all or a portion of a pulmonary ligament 30, a visceral pleura 556, or a mediastinal pleura 38, it is known that biological tissues are pre-stretched or otherwise pre-stressed in vivo for optimal function. For an exemplary embodiment of this particular tissue, the degree of pre-stretch was determined, in at least one method, by measuring the dimensions of the tissue before and after harvest. This was accomplished in this particular example by placing various dots/markings (such as dots/markings 575 shown in FIG. 6F) on the ligament 30, visceral pleura 556 and/or mediastinal pleura 38 itself in its in vivo state to determine the degree of stretch in the two principle directions (referred to herein as the x and y directions). Using such a method, one can characterize that the tissue shrinks by X and Y amount in the x and y directions.

In the glutaraldehyde (or other chemical/mechanism) fixation and mounting process of the tissue on mounts 44 or frames (for stents and other uses as referenced herein), the tissue can be pre-stretched by X and Y to the in vivo values to ensure optimal function of the tissue. In addition, fiber lengths and/or desmosine contents could be measured/obtained in connection with various steps of fixation, including but not limited to determining an amount of tissue shrinkage due to fixation. For example, a stress-strain relation could be determined in fresh lung ligament 30 tissue and processed lung ligament 50 tissue, and a fixed strain could be selected that corresponds to the stress in fresh tissue, for example. Similar stress-strain relations could also be determined in fresh visceral pleura 556 tissue and processed visceral pleura 60 tissue, and/or in fresh mediastinal pleura 38 tissue and processed mediastinal pleura tissue 70. Furthermore, optical means of selection, such as with the use of traditional light, polarized light, and/or other light, with and without magnification, could be used to optically scan the various harvested tissues.

Examples of pulmonary ligament 30, visceral pleura 556 and/or mediastinal pleura 38 harvesting procedure are described as follows. In at least one method relating to harvesting the pulmonary ligament 30 and/or visceral pleura 556, the heart/lung block is extracted from a mammal (such as in connection with a meat processing facility), and the extracted tissue is then be placed in a relatively cold saline solution to help preserve the same. The heart/lung block may be generally referred to herein a pulmonary region tissue, which may include, but is not limited to, lung tissue and one or more of the bronchi, pulmonary artery, pulmonary vein, and/or the heart, so long as the desired tissue to be harvested (pulmonary ligament 30 and/or visceral pleura 556) is contained therein. Similarly, in at least one method relating to harvesting the mediastinal pleura 38, all or the mediastinal portion of the parietal pleura 554 is extracted from a mammal (either through a mediastonaotomy or otherwise), and the extracted tissue is then placed in a relatively cold saline solution to facilitate preservation of the same.

At or before the time of processing, the tissue can be inspected for blood infiltration, fatty material, lymph nodes, perforations, and/or other irregularities, and portions of the tissue containing the same can be treated to either removed the undesired components or discarded/disregarded in view of other portions of the tissue that are relatively homogenous and free of undesired properties, such as perforations or fat.

After selection of desired portions of pulmonary ligament 30, visceral pleura 556 and/or mediastinal pleura 38 from the overall resected tissue, the selected membranes can be mounted in mounts 44 (such as available circular or rectangular frame mounts) and/or fixed with pins or other means of restraint configured to maintain the tissue stretch. Mounting and/or holding the selected tissue in this manner prevents shrinkage and/or folding during fixation. Additionally or alternatively, the tissue can be submerged in a fixation solution (such as glutaraldehyde, for example) for fixation. Prior to mounting and/or fixation, or after mounting and/or fixation if desired, the pulmonary ligament 30, visceral pleura 556, and/or mediastinal pleura 38 can be pre-seeded to make it more likely to endothelialize. As both pulmonary ligament tissue and mediastinal pleura 38 have mesothelium on both sides and visceral pleura has mesothelium on only one side, pre-seeding (also referred to as endothelial seeding) may be performed only on the non-mesothelial side of the visceral pleura 556 tissue and may be skipped altogether with respect to the pulmonary ligament and mediastinal pleura 38 tissues. After fixation, a relatively flat piece of fixed tissue results. Using another method, and after selection of the membranes or desired portions thereof, the membranes can be placed on multidimensional molds, for example, allowing the user to stretch and/or otherwise fit the membrane so as to mimic the mold shape, and then fix the membrane on the mold. With such a method, the resultant fixed material will maintain or closely resemble to multidimensional shape of the mold, and can be used for various purposes.

Various sizes and/or thicknesses of processed lung ligament 50, processed visceral pleura 60, and/or processed mediastinal pleura 70 tissues could be used and be tailored to specific applications. For example, and in embodiments of processed lung ligament 50, processed visceral pleura 60 and/or processed mediastinal pleura 70 tissue ultimately used as valves 400 (exemplary products 100 of the present disclosure), lung ligament 50 tissue of between about 80 microns and about 100 microns to about 300 microns could be used, while visceral pleura 60 between about 40 microns and about 80 microns and/or processed mediastinal pleura 70 having an average thickness between 50 microns and about 80 microns could be used.

Regarding initial tissue harvesting of mediastinal pleura 38 tissue, an exemplary method of the present disclosure includes the step of isolating such tissue from a portion of the mediastinum that separates the right and left lungs (not shown). This portion of the mediastinal pleura 38 in particular tends to be relatively thin, result in a larger surface area available for harvest than other tissues (such as, for example, the pulmonary ligament 30), comprises an abundance of elastin fibers, and exhibits substantial thrombosis resistance and friction resistance integrity. Additionally or alternatively, the method of initially harvesting the mediastinal pleura 38 tissue may include the step of isolating such tissue from the portion of the mediastinal pleura 38 between the pericardium and diaphragm, as such tissue tends to be relatively thin and is typically comprises sufficient surface area to extract leaflet tissue therefrom.

Similarly, with respect to initial tissue harvesting of visceral pleura 556 tissue, an exemplary method of the present disclosure includes the step of isolating tissue 556 from the middle-anterior portion of the lungs, 34 which tends to be relatively thicker and more uniform than other portions of the lung 34. A lateral incision can be made, and using forceps 40 for example, the lung 34 tissue can be carefully pressed away from the visceral pleura 556. FIG. 6F shows a diagram of a portion of a mammalian body 550 showing the lungs 34 and an identified harvest section 552, generally comprising the middle-anterior portion of the lungs 34. Two portions of tissue are shown, namely the parietal pleura 554 that lines the chest cavity and the visceral pleura 556 which lines the lungs 34 and is internal to the parietal pleura 554. As shown in FIG. 6F, and referenced in further detail herein the mesothelial side 860 of visceral pleura 556 is on a relative outside of the lung 34, while the non-mesothelial side 862 of visceral pleura 556 is on the relative inside of the lung 34. Pulmonary region tissue 558 is also shown therein, which may include, but is not limited to, lung tissue and one or more of the bronchi, pulmonary artery, pulmonary vein, and/or the heart, as previously referenced herein. The acquired pulmonary ligament 30 and/or visceral pleura 556 from said pulmonary region tissue may be referred to herein as “samples” of tissue from the pulmonary region tissue 558. After an initial portion of visceral pleura 556 tissue (such as ˜0.5 cm or so) has been separated, the tissue can be gently worked away (manually using one's hand/fingers, for example), taking care/precautions not to overly stress or pull on the visceral pleura 556 tissue. Prior to removal of the visceral pleura 556, the general orientation can be noted and potentially marked on the tissue, noting that visceral pleura 556 has different degrees of potential stretch depending on orientation.

FIG. 6F also demonstrates an exemplary harvesting method whereby pulmonary ligament 30 and/or visceral pleura 556 tissue is harvested and ultimately used in a desired orientation based upon an orientation of harvest. As shown in FIG. 6F, the x-axis (identified as “X” in the figure) may also be referred to herein as a “circumferential” or “transverse” axis or direction/orientation of tissue, and the y-axis (identified as “Y” in the figure) may also be referred to herein as a “vertical” or “axial” axis or direction/orientation of tissue. Natural lung expansion and contraction, consistent with breathing, occurs in a fashion whereby pulmonary ligament 30 and/or visceral pleura 556 would stretch more in the circumferential direction as compared to the axial direction, so whereby pulmonary ligament 30 and/or visceral pleura 556 tissue harvested from a mammal would also have more stretchability in the circumferential direction as compared to the axial direction. Phrased differently, the axial direction is notably stiffer than the circumferential direction, which is also referred to herein as being relatively softer than movement/stretch in the axial direction.

To leverage the inherent non-isotropy of said tissue(s), and using a specific orientation of the same in connection with one or more products 100 of the present disclosure including but not limited to valves 400 as referenced herein, the tissue orientation would be identified at the time of harvest and use accordingly in connection with one or more products 100 of the present disclosure. For example, processed pulmonary ligament 50 and/or processed visceral pleura 60 can be oriented on frame 600, as referenced in further detail herein, so that the axial direction of the product 100 in a mammalian luminal organ (such as a blood vessel) is softer than the circumferential/radial direction, in reference to the product 100, as the circumferential direction is, for example, constrained by the diameter of the blood vessel and cannot distend further, while the axial direction is the direction of opening and closing a valve 400 (in a product 100 embodiment configured as a valve), where more deformation would be needed or desired. Leveraging this non-isotropy (directionality) could be used in connection with various products 100 of the present disclosure depending on the application of interest.

It will be noted that, unlike the orientation-based embodiments described herein with respect to harvesting pulmonary ligament 30 and/or visceral pleura 556, mediastinal pleura 38 can be harvested without attention to orientation. As previously noted, because the collagen fibers in mediastinal pleura 38 do not exhibit significant orientation in a typical direction, mediastinal pleura 38 is mechanically isotropic.

With respect to initial tissue harvesting of pulmonary ligament 30 tissue, an exemplary method of the present disclosure includes the step of isolating tissue 30 from the relative middle section between the lungs 34, as indicated by harvest sections 552 shown in FIG. 6G. The specific harvest section 552 used may depend on mammalian species, the age of the mammal, and/or the thickness of tissue required for a particular application. As with visceral pleura 556 (referenced above and shown in FIG. 6F), pulmonary ligament 30 thickness varies with location within the body. As generally referenced herein, efforts to avoid vascular areas (and/or those areas with blood infiltration), fatty material, lymph nodes, perforations, and/or other irregularities should be made so that desirable pulmonary ligament 30, visceral pleura 556 and/or mediastinal pleura 38 can be obtained. In addition, and regarding certain pulmonary ligament 30 or visceral pleura 556 tissue, avoiding said tissues near the lungs 34 and/or the aorta/esophagus 32 may also lead to preferable pulmonary ligament 30 or visceral pleura 556 harvest. Similar to visceral pleura 556 harvest, and prior to removal of the pulmonary ligament 30, the general orientation can be noted and potentially marked on the tissue, noting that pulmonary ligament 30 has different degrees of potential stretch depending on orientation, noting that as shown in FIG. 6G, ligament 30 is most elastic in the x-direction as shown in the figure.

Pulmonary ligament 30, as referenced herein, may be generally described as a sheet of tissue, and not generally as a combined/bundled tissue. For example, and upon pulmonary ligament 30 harvest, the sections of pulmonary ligament 30 suitable for harvest are generally continuous with the aorta, and are generally not part of the bundled ligament that descends from the mammalian lung root.

General tissue harvesting can apply to several mammalian species, including, but not limited to, cattle, pigs, and horses, such as from blocks of tissue collected after animal slaughter. Harvesting is preferred using clean/sterile conditions, and can proceed after an initial inspection of the blocks of tissue for portions of suitable tissue not having any malformations, abnormalities, perforations, tears, calcifications, spots, etc., as generally referenced herein. The desired tissue (pulmonary ligament 30, visceral pleura 556 or mediastinal pleura 38) can be cleaned using a suitable solution (water and/or saline, for example), and fat and/or muscle covering the tissue can be removed, such as with the use of forceps 40. The removed tissue (pulmonary ligament 30, visceral pleura 556 or mediastinal pleura 38) can be positioned about a mount 44, as described and shown herein, and attached to the same using clamps 42 and/or sutures, such as those comprising Nylon 0, used as overcast stitches, with a needle such as a 333/5 needle. The attachment step can be performed outside of a solution or within a solution, such as a fixative solution. One such fixative solution may comprise 0.65% glutaraldehyde solution BLUE. The dissected tissue can then be stored, upon the mount 44, within an appropriate fixative solution for an appropriate amount of time. For example, and to accomplish initial fixation, the tissue can be fixed in the fixative solution for approximately 24 hours, and the solution can be changed (to either the same fresh fixative solution or to another solution) and stored until the tissues are ready to be cut, formed, manipulated, or otherwise used. Keeping the tissue hydrated is important, as should the tissue completely or partially dry out, it would likely irretrievably lose desired mechanical properties. Long term (or relatively long term) storage can be in, for example, 0.65% glutaraldehyde solution BLUE or another solution for an initial period of time, and then changed to a lower concentration solution (such as 0.50% glutaraldehyde solution CELESTE), for example, and stored until needed.

Regarding fixation, an exemplary fixative solution of the present disclosure can be prepared, resulting in a buffered glutaraldehyde solution, can be prepared as follows. In at least one example of a fixative solution, and in less than 1 L of DDH₂O), the following can be added: 1) 2.05 h of NaOH, 2) 9.08 g of PO₄H₂K, and 3) 13 mL of 50% glutaraldehyde solution (or 26 mL of 25% glutaraldehyde solution). The desired pH would be at or near 7.4 for this exemplary fixative solution, and if the combined solution is not at 7.4, it can be adjusted using additional NaOH solution. After pH adjustment to the desired pH, the overall volume of the flask can be increased to 1.0 L, resulting in the exemplary fixative solution. Other fixative solutions may be optimal for use in connection with various fixation procedures of the present disclosure.

To ultimately fix acquired pulmonary ligament 30, visceral pleura 556 tissue, or mediastinal pleura 38 tissue, at least one fixation method comprises fixing the pulmonary ligament 30, visceral pleura 556 or mediastinal pleura 38 in a fixation solution for at least 24 hours, and optionally at a reduced temperature (such as at or near 23° C.). Other fixation times and temperatures may be used as well. For long-term storage of fixed tissue, storage in 0.5% glutaraldehyde (for example) can protect the fixed tissue. In at least one embodiment, fixation with minimal to no preload is recommended, as preloading may change the mechanical properties of the tissue during and/or after fixation. To maintain preferred tissue fiber orientation, flat or relatively-flat fixation would be recommended, at least with respect to the pulmonary ligament 30 and/or visceral pleura 556. Flat or relatively-flat fixation can be performed, for example, using a tray lined with a silicone elastomer (such as Sylgard), allowing for the tissue to remain flat or relatively flat when pinned down during the fixation process.

Regarding fixation, glutaraldehyde is widely used, and can be used in connection with various buffers, such as HEPES and phosphate buffers. In at least one method, glutaraldehyde is used around a neutral and slightly alkaline pH at or about 7.4, noting that other pH values or ranges can be used with various fixation methods. For example, and in at least one additional fixation method, formaldehyde (formalin) may be used, and/or glycerol may be used. In an exemplary fixation method using glycerol, at or about 98% glycerol may be used to fix the tissue. In at least one embodiment of a method of fixing pulmonary ligament 30, visceral pleura 556 tissue, or mediastinal pleura 38 tissue, bovine serum albumin (BSA) can be used to remove cytotoxicity in connection with fixation, such as fixation using glutaraldehyde and/or formaldehyde. Eliminating glutaraldehyde and/or formaldehyde from the storage solution may be beneficial as such compositions are quite cytotoxic, and storage of fixed tissue in non-toxic solutions or using dry tissue technologies can be useful to stored said fixed tissue for various amounts of time.

Other fixation methods may include, but are not limited to, various cryo-preservation or dry tissue fixation methods known are developed in the art for tissue fixation. Furthermore, fixation could be performed at various loads or strains, such as in vivo stretch ratios, as determined by the markers (dots placed upon the tissue prior to harvest). For example, and as referenced above and at the time of or prior to harvest, markings could be placed on the lung ligament 30, visceral pleura 556 tissue or mediastinal pleura 38 (using a marker, for example), and measurements between markings could provide the harvester with information relating to said tissue at a natural (non-stretched state). When placing said harvested tissue upon a frame for fixation, for example, said tissue could be stretched at various degrees of stretch, with either raw distance stretch being known and/or a percentage stretch being known based upon the distance between markings at the natural (non-stretched) and stretched states.

With respect to overall pulmonary ligament 30, visceral pleura 556 and/or mediastinal pleura 38 preparation, preservation of the tissue's elastin component is important so that the intended uses of the prepared pulmonary ligament 50, processed visceral pleura 60, and/or processed mediastinal pleura 70 may still be considered. As the overall flexibility of the processed pulmonary ligament 50, processed visceral pleura 60 and/or processed mediastinal pleura 70 preparation is important for various uses, efforts to preserve the elastin component may be reflected in the overall preparation methods. Different methods may be used to generate different products 100 of the present disclosure, such as different frames, means of restraint, tissue stretching, fixation duration, and/or a combination of the same. Furthermore, decellularization of the epithelial layer or layers of pulmonary ligament 30, for example, can be performed while also preserving/keeping the elastin scaffolds. As is known, the biologically occurring pulmonary ligament and mediastinal pleura 38 both include a layer of mesothelial cells (a specialized type of epithelial cells) on each side of the ligament and pleura, respectively. In addition, storage can be had using saline and/or an additional preservative, so that the product 100 is safe to use when needed.

Pulmonary ligaments 30, visceral pleura 556, and/or mediastinal pleura 38 tissue for potential use in connection with the present disclosure can be harvested from any number of mammalian species and used in the same or other species. For example, pulmonary ligaments 30, visceral pleura 556, and/or mediastinal pleura 38 tissue can be harvested from pigs, horses, cows, goats, sheep, etc. and used to treat the same species or different species, including humans. Further, pulmonary ligaments 30, visceral pleura 556 and/or mediastinal pleura 38 tissue could be harvested from one human and used to treat another human. For long or short term storage, for example, pulmonary ligaments 30, visceral pleura 556 and/or mediastinal pleura 38 (and/or processed ligament 50, processed visceral pleura 60, processed mediastinal pleura 70 and/or products 100 of the present disclosure) may be preserved by freezing in liquid nitrogen (−198° C. in at least one example). To ensure that fixed tissue thickness, stiffness, strength, and/or micro-structure do not change (or substantially change) over time, various short- and/or long-term storage mechanisms may be used.

In at least one embodiment of a method of preparing fixed/processed lung ligament 30, visceral pleura 556 tissue and/or mediastinal pleura 38 tissue of the present disclosure, the method includes the steps of obtaining a heart/lung block and/or thoracic cavity (such as from a slaughterhouse), placing the heart/lung block and/or thoracic cavity in cold saline (or another suitable solution at various temperatures) for transport as needed, isolating the lung ligament 30, visceral pleura 556 tissue and/or mediastinal pleura 38 tissue, and fixing the same as referenced herein. Such a method may be performed while taking precautions/steps to avoid tissue, perforations, fenestrations, and/or blood vessels or infiltrations therein.

In at least one embodiment of a product of the present disclosure, the product is not treated with a fixative. Instead, the product, in at least one embodiment, is harvested from a mammal and used in connection with one or more procedures or as one or more products 100 reference herein without the use of a fixative. In certain aspects, such non-fixed pulmonary ligament and/or mediastinal pleura products can be acellular (e.g., after treatment with one or more decellularization agents) and/or sterile.

In additional embodiments, provided are medical articles (exemplary products 100), such as kits 525, shown in block diagram form in FIG. 6E, that may include processed pulmonary ligament 50, processed visceral pleura 60 tissue, processed mediastinal pleura 70 tissue and/or a product 100, sterilely enclosed within packaging 530. A sterile condition of pulmonary ligament 50, processed visceral pleura 60 tissue, processed mediastinal pleura 70 tissue and/or a product 100 within the packaging 525 may be achieved, for example, by terminal sterilization using irradiation, ethylene oxide gas, or any other suitable sterilization technique, and the materials and other properties of the medical packaging can be selected accordingly.

Uses of a processed pulmonary ligament 50, processed visceral pleura 60, and/or processed mediastinal pleura 70 tissue as referenced above, include, but are not limited to, the following applications:

a. As a cover for various stents, such for as coronary stents, peripheral stents (porto cava shunts), aortic stents, neurological stents, esophageal stents, biliary tract stents, and the like.

b. As various types of biological tissue valves, including, but not limited to, venous and/or arterial valves, which may have various leaflet configurations, such as monocusp, bileaflet, trileaflet, and others.

c. As a cover for saphenous vein bypasses, thus avoiding vein over-distension.

d. As a patch, in various cardiac and other surgical procedures, such as ventricular reconstruction, an arterial patch, a venous patch (such as a carotid endarterectomy), or to repair other holes.

e. As a placement around the ascending aorta after surgery to avoid aortic aneurysm formation in hypertensive patients.

f. As a membrane in cardiac, thoracic, or general surgery to avoid adhesion in reoperations (valvular, transplants, left ventricular assist device (LVAD), coronary artery bypass graft (CABG), pediatric surgery, or general surgery).

g. As a cover for LVAD diaphragms or a total artificial heart diaphragm.

h. As a cover for the synthetic net in hernia repair and abdominal dehiscense.

i. As a biologic skin substitute in burn patients avoiding infection and loss of proteins, water.

j. As a cover for organs such as the heart (to limit dilation of the left ventricle, for example), stomach, urinary bladder, and to avoid overdistension and/or to prevent adhesion especially in laparoscopic procedures of diabetic patients.

k. As a reinforcement of a suture line, such as with ventricular aneurysm repair, bariatric surgery, and fistulae repair for intestines, bronchus, and esophagus.

l. As a structure for biological composite tubes, such as stented or stentless valves for inclusion within a biological tube, which can be used, for example, in ascending aortic aneurysm (AAA) replacement or pulmonary artery replacement.

m. In orthopedic surgery, such as with tendon replacement (having advantages in resistance and elasticity), total or partial replacement of the articular capsulae during surgery of the hip, elbow, knee, and/or the like, and/or as a cover for various orthopedic prosthetic devices.

n. As a cover for neurosurgical applications, such as a cover of part of the brain surface during tumor resection or resection of the skull.

o. In urologic surgery, such as in connection with reconstruction of a partial or total urinary bladder and/or urethral resection.

p. In gynecological surgery, such as in connection with vaginal reconstruction after tumor resection or other trauma, with reconstruction of perineal muscles to fix the urinary bladder, or with uterus prolapse.

q. In head & neck surgery, such as in connection with reconstructive surgery, replacement of muscles (requiring elasticity and resistance), and as a cover for a maxillary prosthesis.

r. In connection with other trauma, such as treating vehicular accident victims by covering complex wounds until surgical repair, which may be complex, can take place.

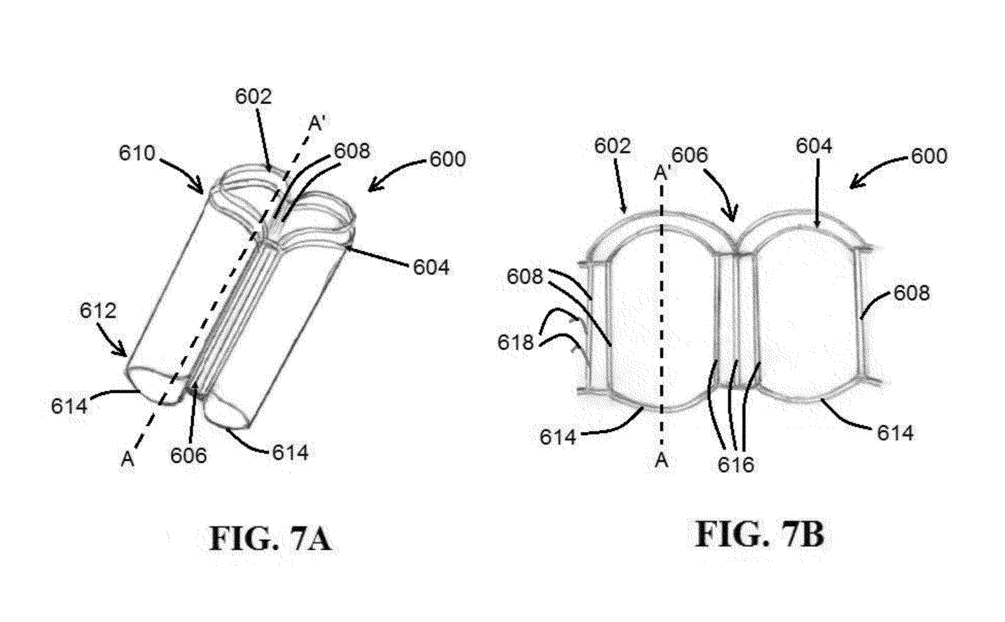

In view of the various uses of processed pulmonary ligament 50, processed visceral pleura 60, and/or processed mediastinal pleura 70 to create various products 100 of the present disclosure, said ligament 50, visceral pleura 60 and/or mediastinal pleura 70 may be used to produce products 100 configured as stents and/or stent valves 400 as follows. FIGS. 7A and 7B show closed and opened stent valve frames, respectively, for use with various products 100 of the present disclosure. As shown in FIGS. 7A and 6B, an exemplary product 100 of the present disclosure comprises a frame 600, with said frames 600, in various embodiments, comprising at least one superior arm 602 and at least one inferior arm 604. Arms 602, 604, as shown in FIGS. 7A and 7B, may be positioned at or near a relative end of frame 600, and may be parallel or substantially parallel to one another. Frames 600, as shown in FIGS. 7A and 7B, further comprise a connection portion 606, and optionally one or more vertical bars 608 extending along an elongate axis (A-A′ as shown therein) to provide additional overall stability. As shown in FIG. 7B, an exemplary frame 600 comprises three vertical bars 608 extending along axis A-A′ along a portion of a length of frame 600 from a first end 610 to a second end 612.

At or near the relative second end 612 of frame 600, one or more lower arms 614 may be present, which may, as shown in FIG. 7B, connect to one or more vertical bars 608 and/or one or more elements of connection portion 606. A combination of vertical bars 608, as referenced in further detail below, may comprise a connection portion 606. Frames 600, or portions thereof, may comprise a number of biologically-compatible materials including, but not limited to, nitinol, chromium, cadmium, molybdenum, nickel, a nickel composite (such as, for example, nickel-cadmium and/or nickel-chromium), nitinol palladium, palladium, cobalt, platinum, and/or stainless steel.

Connection portion 606 is shown in FIG. 7B as being an element of an exemplary frame 600 coupling to one or more of superior arm(s) 602, inferior arm(s) 604, vertical bar(s) 608, and lower arm(s) 614. In at least one embodiment, and as shown in FIG. 7B, connection portion 606 comprises a plurality of connection bars 616, which are used to connect one or more processed ligaments 50 and/or visceral pleura 60, or one or more other bodily tissues having the necessary stretchability and durability properties necessary to be useful in connection with one or more products 100 of the present disclosure, to frame 600 as referenced in further detail herein. As referenced herein, a “tissue” may be referred to as a ligament 50, visceral pleura 60, and/or mediastinal pleura 70, and ligament 50, visceral pleura 60, and/or mediastinal pleura 70 in at least one embodiment may comprise another non-ligament tissue having the necessary properties noted above.

As noted above, a plurality of vertical bars 608 may also comprise a connection portion 606 of the present disclosure. Therefore, and depending on how portions of frame 600 are viewed, the exemplary frame shown in FIGS. 7A and 7B may comprise one connection portion 606 and a plurality of vertical bars 606, or they may comprise two connection portions 606, with one connection portion 606 comprising connection bars 616 and the other connection portion 606 comprising vertical bars 608. In addition, and as shown in FIG. 7B, various frames 600 of the present disclosure may comprise one or more barbs 618 positioned along various portions of frames 600 (such as vertical bars 608, connection bars 616, and/or other components) to facilitate securing a product 100 within a mammalian luminal organ (to prevent migration), and/or to facilitate securing the tissue (such as ligament 50, visceral pleura 60, and/or mediastinal pleura 70) to frame 600.

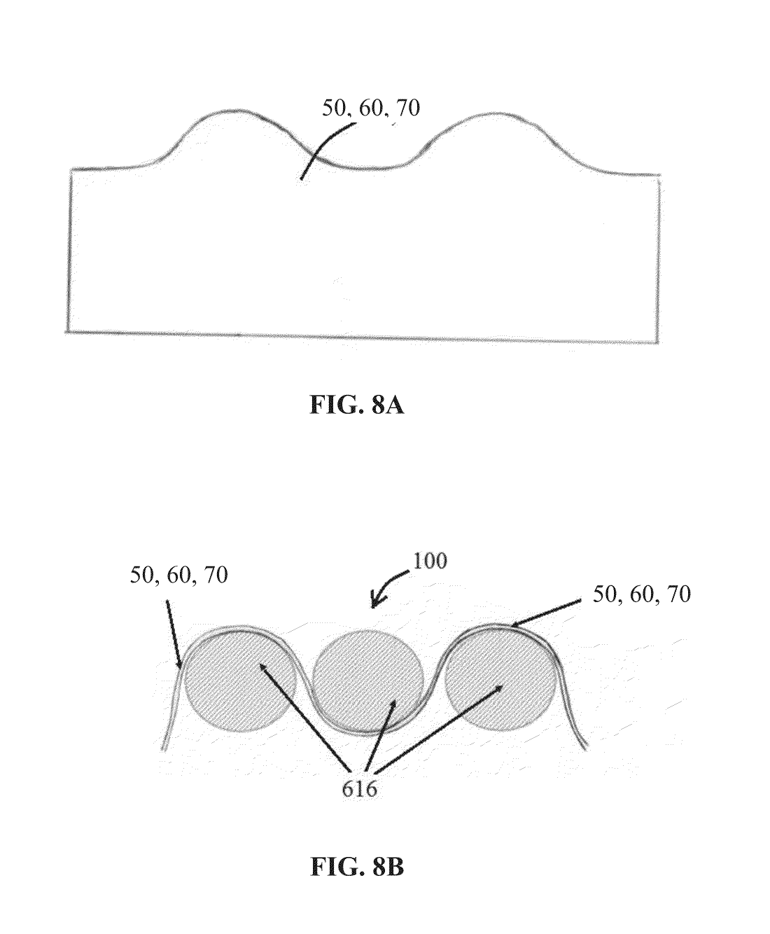

FIG. 8A shows an exemplary processed ligament 50, visceral pleura 60 and/or mediastinal pleura 70 of the present disclosure molded for use with or as a bileaflet valve 400. As shown in FIGS. 7A and 7B, frame 600 is configured as two leaves with one connection portion 606. As referenced further herein, other frame 600 embodiments, such as being configured as a trileaflet valve 400 and as potentially a valve 400 with even more leaflets, may be produced consistent with the present disclosure. The processed ligament 50, visceral pleura 60 and/or mediastinal pleura 70, as shown in FIG. 8A, is shaped substantially similar to an outer perimeter of frame 600 shown in FIGS. 7A and 7B. The shape shown in FIG. 8A represents processed ligament 50, visceral pleura 60, and/or mediastinal pleura 70 configured so to create symmetrical valve leaflets upon placement of processed ligament 50, visceral pleura 60 and/or mediastinal pleura 70 upon frame 600.

FIG. 8B shows a cross-section of a portion of an exemplary product 100 of the present disclosure, whereby individual connection bars 616 of an exemplary frame 600 are shown with a portion of a processed ligament 50, visceral pleura 60, and/or mediastinal pleura 70 positioned therebetween. This view may be considered as an upper or lower cross-sectional view, and demonstrates an exemplary method of positioning a portion of processed ligament 50, visceral pleura 60, and/or mediastinal pleura 70 within said connection bars 616 to secure the processed ligament 50, visceral pleura 60, and/or mediastinal pleura 70 at that particular location within device 100.

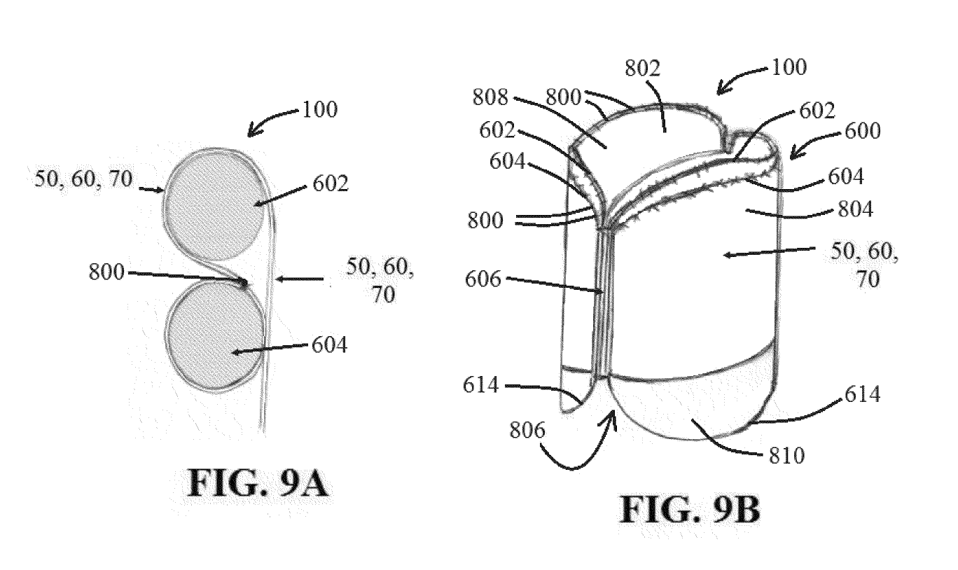

FIG. 9A shows another cross-section of a portion of an exemplary product 100 of the present disclosure, whereby a superior arm 602 and an inferior arm 604 of an exemplary frame 600 are shown with a portion of a processed ligament 50, visceral pleura 60, and/or mediastinal pleura 70 positioned therebetween. This view shows an exemplary method of positioning a portion of processed ligament 50, visceral pleura 60, and/or mediastinal pleura 70 within said arms 602, 604 to secure the processed ligament 50, visceral pleura 60, and/or mediastinal pleura 70 at that particular location within device 100. One or more sutures 800, as shown in FIG. 9A, may be used to connect two portions of processed ligament 50, visceral pleura 60, and/or mediastinal pleura 70 to one another to prevent movement of the same. For example, and as shown therein, an end portion of processed ligament 50, visceral pleura 60, and/or mediastinal pleura 70 may be positioned upon or adjacent to inferior arm 604, and wrapped counter-clockwise (as shown in FIG. 9A) around inferior arm 604. When the wrapped portion of processed ligament 50 and/or visceral pleura 60 is positioned near the end portion, it may continue being wrapped around frame 600 by way of wrapping clockwise (as shown in FIG. 9A) around superior arm 602, and the processed ligament 50, visceral pleura 60, and/or mediastinal pleura 70 may be sutured to itself as shown in the figure.

An exemplary embodiment of a product 100 of the present disclosure comprising a frame 600 and processed ligament 50, visceral pleura 60 and/or mediastinal pleura 70 attached thereto is shown in FIG. 9B. Product 100 is shown in a closed configuration in FIG. 9B, whereby processed ligament 50, visceral pleura 60 and/or mediastinal pleura 70 is sutured to itself and/or to portions of frame 600 at multiple locations to hold the processed ligament 50, visceral pleura 60 and/or mediastinal pleura 70 in place. As shown in FIG. 9B, product 100 is configured as a bileaflet valve 400, which may be used, for example, as a venous valve or another type of valve. Leaflets 802 and 804 are identified in FIG. 9B. In various embodiments referenced herein, processed ligament 50, visceral pleura 60, and/or mediastinal pleura 70 is sutured to frame 600, but sutures 800 are outside of the bloodstream (not in contact with blood flow) when frame 600 with processed ligament 50, visceral pleura 60, and/or mediastinal pleura 70 thereon (an exemplary product 100) is positioned within a mammalian luminal organ having blood flowing therethrough.

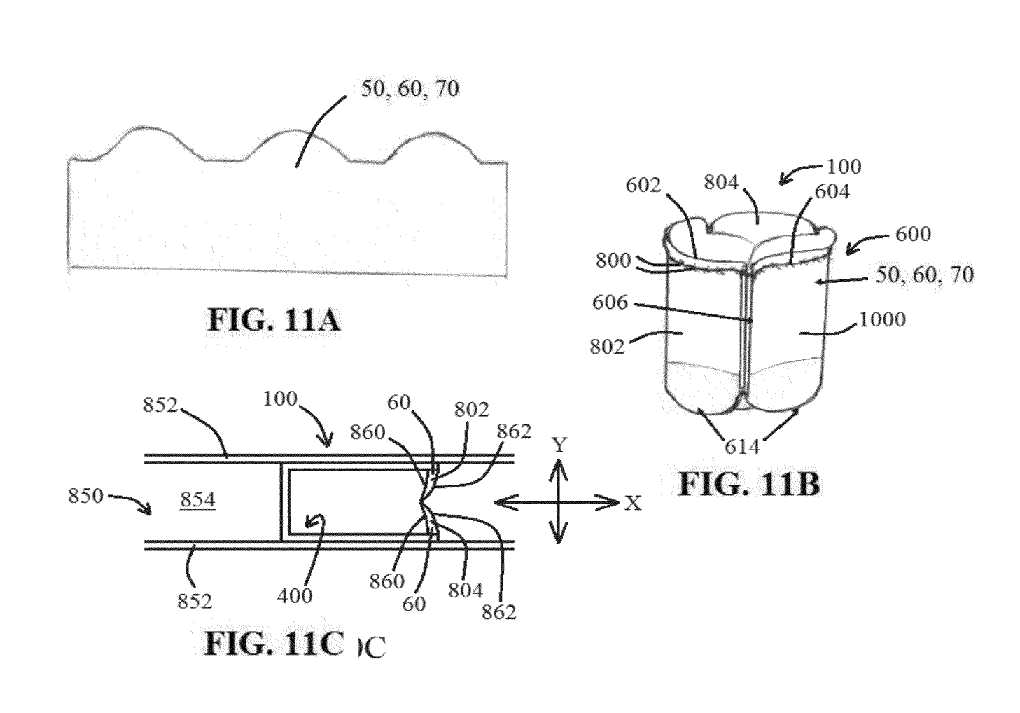

A completed product 100 (such as shown in FIG. 9B and in FIG. 11B described below) may be configured as a stent or stent valve 400. Configurations as a stent valve 400 would utilize leaflets 802 and 804 to control the flow of fluid through a lumen 806 defined within product 100. The direction of fluid flow of such an embodiment would be such that fluid would enter inlet portion 808 of product 100 and exit from outlet portion 810 of product 100, as shown in FIG. 9B. In such a configuration, product 100 could be positioned within a mammalian luminal organ, and fluid flow through said organ could continue through lumen 806 of product 100.

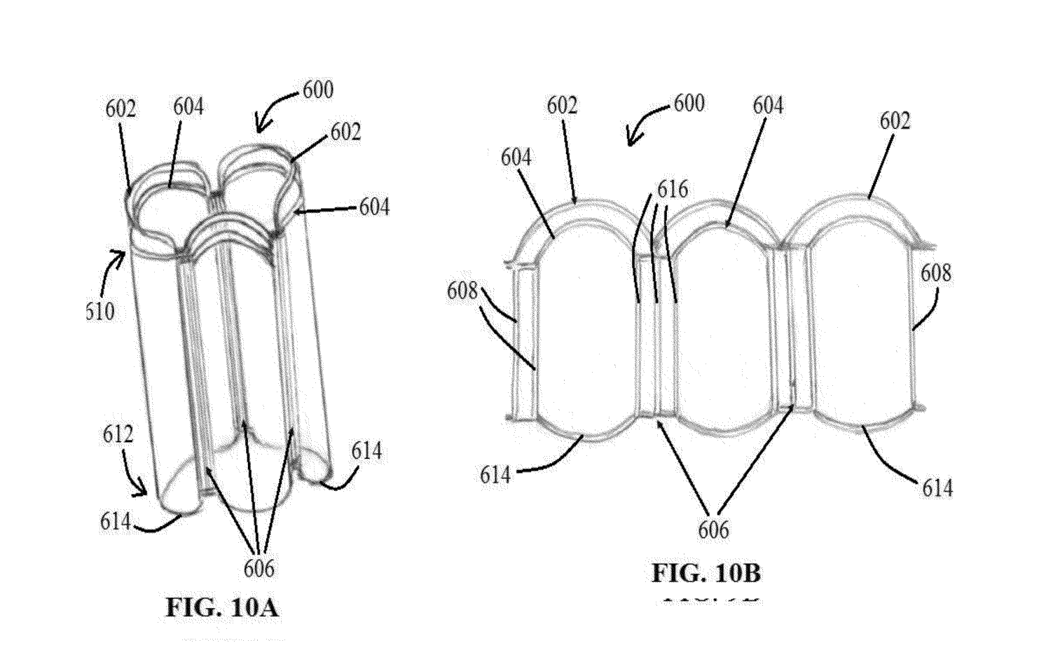

FIGS. 10A and 10B show additional exemplary closed and opened stent valve frames, respectively, for use with various products 100 of the present disclosure. As shown in FIGS. 10A and 10B, an exemplary product 100 of the present disclosure comprises a frame 600 configured for ultimate use as a trileaflet valve 400, with said frames 600, in various embodiments, comprising at least one superior arm 602 and at least one inferior arm 604. Arms 602, 604, as shown in FIGS. 10A and 10B, may be positioned at or near a relative end of frame 600. Frames 600, as shown in FIGS. 10A and 7B, further comprise two or more connection portions 606 (as referenced in further detail below), and optionally one or more vertical bars 608 extending along an elongate axis to provide additional overall stability. As shown in FIG. 10B, such an exemplary frame comprises three vertical bars 608 extending along a portion of a length of frame 600 from a first end 610 to a second end 612. At or near the relative second end 612 of frame 600, one or more lower arms 614 may be present, which may, as shown in FIG. 7B, connect to one or more vertical bars 608 and/or one or more elements of connection portion 606. A combination of vertical bars 608, as referenced herein, may comprise a connection portion 606.

Depending on how portions of frame 600 are viewed, the exemplary frame 600 shown in FIGS. 10A and 10B may comprise two connection portions 606 and a plurality of vertical bars 608, or they may comprise three connection portions 606, with two connection portions 606 comprising connection bars 616 and the other connection portion 606 comprising vertical bars 608. FIG. 10A shows frame 600 as having three connection portions 606, while the same frame 600, shown in FIG. 10B, shows two connection portions 606 and a plurality of vertical bars 608. The frames shown in FIGS. 10A and 10B are identical, however, with one being shown in a closed configuration (FIG. 10A) and the other being shown in a closed configuration (FIG. 10B).

Connection portions 606 are shown in FIG. 10B, for example, as being elements of an exemplary frame 600 coupling to one or more of superior arm(s) 602, inferior arm(s) 604, vertical bar(s) 608, and lower arm(s) 614. In at least one embodiment, and as shown in FIG. 10B, connection portions 606 comprise a plurality of connection bars 616, which are used to connect one or more processed ligaments 50, visceral pleura 60, and/or mediastinal pleura 70 to frame 600 as referenced herein with respect to other frame 600 and/or product 100 embodiments.

FIG. 11A shows an exemplary processed ligament 50, visceral pleura 60, and/or mediastinal pleura 70 of the present disclosure molded for use with as a trileaflet valve 400. As shown in FIGS. 10A and 10B, frame 600 is configured as three leaves with two or three connection portions 606, depending on how the frame 600 is viewed. The processed ligament 50, visceral pleura 60, and/or mediastinal pleura 70, as shown in FIG. 11A, is shaped substantially similar to an outer perimeter of frame 600 shown in FIGS. 10A and 10B. The shape shown in FIG. 8A represents processed ligament 50, visceral pleura 60, and/or mediastinal pleura 70 configured so to create symmetrical valve leaflets upon placement of processed ligament 50, visceral pleura 60, and/or mediastinal pleura 70 upon frame 600.

An exemplary embodiment of a product 100 of the present disclosure comprising a frame 600 as shown in FIGS. 10A and 10B and a processed ligament 50, visceral pleura 60, and/or mediastinal pleura 70 attached thereto is shown in FIG. 11B. Product 100 is shown in a closed configuration in FIG. 11B, whereby processed ligament 50, visceral pleura 60 and/or mediastinal pleura 70 is sutured to itself and/or to portions of frame 600 at multiple locations to hold the processed ligament 50 and/or visceral pleura 60 in place. As shown in FIG. 11B, product 100 is configured as a trileaflet valve 400, which may be used, for example, as a venous valve or another type of valve. Leaflets 802, 804, and 1000 are identified in FIG. 11B.

Various products 100 of the present disclosure configured as valves 400, including products 100 shown in FIGS. 9B and 11B for example, and/or other valve 400 products of the present disclosure used with or without various frames, can have the processed ligament 50, visceral pleura 60, and/or mediastinal pleura 70 positioned in specific configuration(s) to improve overall operation, effectiveness, and/or size of said products 100. Visceral pleura 556, and therefore processed visceral pleura 60, has one side with mesothelium (also referred to herein as a relatively smooth “mesothelial side”), and has an opposite side without mesothelium (also referred to herein as a relatively rough “non-mesothelial side”). As shown in FIG. 6F, the mesothelial side 860 of visceral pleura 556 is on a relative outside of the lung 34, while the non-mesothelial side 862 of visceral pleura 556 is on the relative inside of the lung 34.

For example, and in connection with various products 100 of the present disclosure using processed visceral pleura 60 as one or more valve 400 leaflets 802, 804, or 1000, processed visceral pleura 60 can be positioned in a way/configuration so that the side of processed visceral pleura 60 having mesothelium would be on the relative back of the valve 400 leaflet(s) 802, 804, and/or 1000, and so that the side of processed visceral pleura 60 without mesothelium would be on the relative front of the valve 400 leaflet(s) 802, 804, and/or 1000. In such a configuration, the mesothelial side 860 of processed visceral pleura 60 is on the back of leaflet(s) 802, 804, and/or 1000, where blood flow reversal exists as the valve 400 closes. The relatively smooth mesothelial side 860 would be in contact with blood flows more slowly, where shear stresses may be lower and reversing. As such, the rougher non-mesothelial 862 side of processed visceral pleura 60 would then be on the front of leaflet(s) 802, 804, and/or 1000, in contact with fast moving blood, because there is less of a risk of thrombosis as compared with the slower moving blood or shear stress.

Such a valve 400 (exemplary product 100) embodiment is shown in FIG. 11C positioned within a luminal organ 850, where valve leaflets 802, 804 (or more, less, or different leaflets, depending on valve 400 configuration) are shown therein. As shown therein, valve 400 is in contact with the wall(s) 852 of luminal organ 850, positioned within a lumen 854 defined therethrough. A mesothelial side 860 of processed visceral pleura 60 is on a relative back of leaflets 802, 804, and a non-mesothelial side 862 of processed visceral pleura 60 is on a relative front of leaflets 802, 804, as described above. Such a device embodiment 100 is one such embodiment referenced herein where processed ligament 50, visceral pleura 60, and/or mediastinal pleura 70 is/are oriented in a specific direction. In at least another embodiment, for example, mesothelial side 860 of processed visceral pleura 60 would be on a relative front of leaflets 802, 804, and a non-mesothelial side 862 of processed visceral pleura 60 would be on a relative back of leaflets 802, 804.

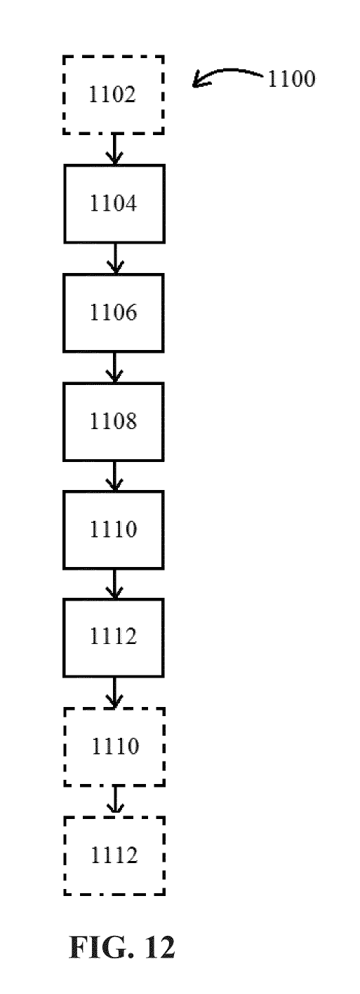

Exemplary products 100 of the present disclosure may be prepared as follows. In at least one method for preparing a product of the present disclosure, the method 1100, as shown by the method steps in FIG. 12, comprises the steps of preparing a bodily tissue (such as a processed ligament 50, visceral pleura 60, and/or mediastinal pleura 70 or another bodily tissue having the necessary stretchability and durability properties necessary to be useful in connection with one or more products 100 of the present disclosure) (an exemplary tissue preparation step 1102, which may be optional, as the tissue may have been previously prepared and subsequently used in connection with method 1100), and shaping the bodily tissue (an exemplary tissue shaping step 1104) so that the tissue will fit around portions of an exemplary frame 600. In at least one embodiment, tissue preparation step comprises preparing a portion of tissue (such as pulmonary ligament 50, visceral pleura 60, and/or mediastinal pleura 70) by way of excising the tissue from a mammalian body, removing any undesirable portions of tissue (such as those with holes, lymph nodes or vessels therein), placing the tissue on a frame (to maintain a desired shape and/or amount of stretch), and fixing the tissue using glutaraldehyde and buffer, for example. Tissue shaping step 1104, in at least one embodiment, comprises stretching the tissue (such as lung ligament 50, visceral pleura 60, lung viscera, mediastinal pleura 70, and/or another tissue) and cutting the tissue using a flat mold, for example.

In various embodiments, method 1100 further comprises the step of positioning the tissue around a mount (such as a cylindrical or conical mount, which may be made of acrylic or another suitable material) (an exemplary mounting step 1106), and positioning at least part of an exemplary frame 600 around the tissue positioned upon the mount (an exemplary frame positioning step 1108). Tissue may then be passed around various bars of frame 600 (such as connection bars 616 of connection portion 606 or other components of frame 600), such as shown in FIG. 8B (an exemplary tissue connection step 1110), and various sutures 800 may be used to suture portions of tissue together to form the overall relatively cylindrical shape (an exemplary suturing step 1112). Tissue connection step 1110 may be repeated, such as by allowing the inflow portion of the tissue cylinder to pass through the superior and inferior parallel arms (arms 602, 604) to cover arms 602, 604, as shown in FIG. 9A, for example. Additional sutures may then be used, by way of repeating suturing step 1112, so that the border of the tissue is sutured with, for example, a continuing suture line facilitated by using a polypropylene 7-0 or 8-0 needle, for example, or another type/size of needle, to result in a product 100 as shown in FIGS. 9B, 11B, or in other product 100 embodiments.

After product 100 is prepared, it can be delivered into a mammalian luminal organ in a number of ways. One method of delivery involves gently crimping or compressing product 100 so that its overall cross-section decreases, to facilitate delivery into the luminal organ. This delivery may be facilitated using a catheter or a wire, for example. If delivered by catheter, and it at least one embodiment (such as with a nickel-cadmium product 100 of the present disclosure), a balloon catheter may be used, with product 100 positioned at the balloon. Inflation of the balloon, using a gas or a liquid, for example, can cause the balloon to expand and thus cause product 100 to expand and be positioned within the luminal organ. Deflation of the balloon can then facilitate removal of the catheter. Furthermore, products 100 of the present disclosure may be autoexpandable, such as those comprising nitinol, whereby delivery using a balloon catheter may not be necessary. Delivery of products 100 of the present disclosure is not limited to the aforementioned delivery methods, as other methods of delivering implantable devices into a mammalian luminal organ may be used to deliver products 100.

The present disclosure also includes disclosure of uses of various processed ligaments 50, processed visceral pleura 60, mediastinal pleura 70 and/or products 100 in connection with various Transcatheter Aortic-Valve Implantation (TAVI) and other percutaneous approaches. TAVI involves the placement of an aortic valve within a patient using a catheter to avoid a traditional open surgical procedure and to minimize general stresses to the patient during the procedure. This procedure is used when a patient's aortic valve fails to operate as desired, and can effectively prolong the patient's life without requiring additional surgical and non-surgical procedures, including but not limited to heart transplant. Certain patients may not be suitable for surgery, such as those with such a severe aortic stenosis that would preclude an open surgical procedure, allowing TAVI to be considered. Processed ligaments 50, processed visceral pleura 60, and/or mediastinal pleura 70 of the present disclosure can be used with current or potentially developed aortic valve frames/housings, or products 100 of the present disclosure comprising one or more frames 600, can be used as aortic or other valves as referenced herein. Furthermore, various processed ligaments 50, processed visceral pleura 60, processed mediastinal pleura 70 and/or products 100 can be delivered percutaneously or surgically, using various catheters or wires or other surgical tools for example, avoiding more invasive surgical procedures.

As processed lung ligaments 50, processed visceral pleura 60, and processed mediastinal pleura 70 of the present disclosure are thinner than pericardium, which is currently used in TAVI or used with any number of valve procedures to replace and/or insert various aortic, mitral, pulmonary, tricuspid, and/or other percutaneous valves, the overall dimensions of the final delivery system, whether it be a product 100 of the present disclosure or processed ligament 50, processed visceral pleura 60, and/or mediastinal pleura 70 of the present disclosure coupled with another type of frame or housing, can be significantly reduced by using processed ligaments 50, processed visceral pleura 60, and/or mediastinal pleura 70 instead of pericardium. The bulk of a traditional TAVI product is not the stent frame itself, but the pericardial tissue, and using processed ligament 50, processed visceral pleura 60, and/or mediastinal pleura 70 of the present disclosure instead of pericardial tissue would notably and beneficially decrease the overall bulk of said product 100. Having a product 100 configured smaller than a traditional TAVI product, for example, would not only allow for more potential manipulation of said product 100 in connection with delivery, expansion, and/or placement as compared to traditional products, but also would allow for smaller delivery devices (catheters, for example) to be used, therefore decreasing the potential aperture/opening made into a femoral or iliac artery, for example, during product 100 delivery. For example, reducing a catheter from 18 French to 12 French, or from 12 French to 8 French, would permit a smaller delivery aperture/opening to be used. This would also reduce or eliminate the need for a potential closure device, reduce patient bleeding, reduce overall patient trauma, and/or simplify delivery, placement, and/or expansion of relatively smaller products 100.

There are various advantages to using products 100 of the present disclosure as valves and/or as other medical implantable devices. For example, and with various embodiments described herein, products 100 are configured to avoid suture of commissure and thus spread out the stress, and there is may be no sutures 800 that come in contact with blood. Frames 600 may have a less metallic stent design, and may also comprise a completed inflow metal stent tissue cover. With respect to the different product 100 borders, various products 100 of the present disclosure have no suture line at the inflow border, and no tissue (such as processed lung ligament 50, visceral pleura 60, and/or mediastinal pleura 70) fixation at the stent border. The double parallel (or relatively/substantially parallel) arms (superior arm(s) 602 and inferior arm(s) 604)) are configured so that a tissue (such as ligament 50, visceral pleura 60, and/or mediastinal pleura 70) can be passed around them. Furthermore, and in at least one product 100 embodiment the suture line is not submitted to the inflow stress and blood flow, and the suture knot is not in contact with the inflow blood.

Other advantages of products 100 of the present disclosure also exist. For example, and when preparing said products 100, the commissure are obtained by passing the tissue around the various vertical arms with the advantage of no suture and diffuse tissue stress along the vertical length of the bars. The various frame 600 designs and their tissue covers have the advantage of very little contact of any metallic frame material with the blood flow. The valves themselves have excellent leaflet coaptation, good valve sinus formation, and no blood stagnation areas when developed/configured as described herein and used within a mammalian blood vessel. Furthermore, the inflow stent area covered with tissue is in broad contact with the venous wall with the advantage of tissue-tissue contact when positioned within a mammalian vein.

While various embodiments of biological tissue products and methods of using and generating the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A method of processing a tissue, the method comprising the steps of:
acquiring a tissue comprising at least a portion of parietal pleura tissue from a mammal;
selecting a sample of mediastinal pleura tissue from the parietal pleura tissue; and
fixing the sample of mediastinal pleura tissue using a fixative, resulting in a fixed sample.

2. The method of claim 1, wherein the step of acquiring comprises acquiring the parietal pleura by way of dissecting or resecting tissue from a deceased mammal.

3. The method of claim 1, wherein the step of acquiring comprises acquiring the at least a portion of parietal pleura tissue from a mammal selected from the group consisting of a pig, a horse, a cow, a goat, a sheep, and a human.

4. The method of claim 1, wherein the step of selecting a sample of mediastinal pleura tissue further comprises isolating and harvesting a sample of mediastinal pleura tissue from a section of the parietal pleura tissue positioned between a right lung and a left lung of the mammal.

5. The method of claim 1, wherein the step of selecting a sample of mediastinal pleura tissue further comprises isolating and harvesting a sample of mediastinal pleura tissue from a section of the parietal pleura tissue that extends between a pericardium and a diaphragm of the mammal.

6. The method of claim 1, wherein the step of selecting a sample of mediastinal tissue from the parietal pleura tissue further comprises the step of removing lymph nodes and fatty material from the portion of parietal pleura tissue.

7. The method of claim 1, wherein the method further comprises the step of:
placing the sample of mediastinal pleura tissue within or upon a mount having known dimensions.

8. The method of claim 1, further comprising the step of:
forming the fixed sample of mediastinal pleura tissue into a product;
wherein the product is selected from the group consisting of a stent cover, a diaphragm cover, a hernia repair cover, a brain cover, a general organ cover, a wound cover, a prosthetic device cover, a skull cover, a general tissue cover, a valve, a patch, a surgical membrane, a skin substitute, a suture reinforcement, a tubular structure, a tendon replacement, a bladder tissue replacement, a urethra tissue replacement, a vaginal tissue replacement, and a muscle replacement.

9. The method of claim 8, wherein the product comprises a first surface and a second surface and the composition of the product includes elastin fibers;
wherein the first and second surfaces of the product are lined with mesothelial cells.

10. The method of claim 1, further comprising the steps of:
positioning the fixed sample of mediastinal pleura tissue upon at least a portion of a frame, wherein the fixed sample of mediastinal pleura tissue and the frame collectively form a tissue product; and
positioning the tissue product within a mammalian lumen so that fluid native to the mammalian lumen may pass through a lumen defined within the tissue product.

11. The method of claim 10, wherein the tissue product comprises a valve having a leaflet configuration.

12. The method of claim 11, wherein the valve has a bileaflet configuration or a trileaflet configuration.

13. The method of claim 10, wherein the fixed sample of mediastinal pleura tissue comprises tissue having stretchability and durability properties sufficient to allow the fixed sample of mediastinal pleura tissue to move relative to the fluid flow through the lumen defined within the tissue product.

14. The method of claim 1, further comprising the step of:
decellularizing at least a portion of the sample of the mediastinal pleura tissue prior to performing the fixing step.

15. The method of claim 1, further comprising the step of:
treating a patient using the fixed sample of mediastinal pleura tissue.

16. A tissue product, the product comprising:
a frame configured to retain a mammalian tissue thereon; and
the mammalian tissue coupled to the frame;
wherein the mammalian tissue comprises mediastinal pleura tissue and the product is configured such when the product is positioned within a mammalian lumen, fluid native to the mammalian lumen may pass through a lumen defined within the product.

17. The tissue product of claim 16, wherein the product is configured for mammalian treatment or therapy.

18. The tissue product of claim 16, wherein the product comprises an outer surface and an intraluminal surface and the composition of the product includes elastin fibers;
wherein the outer and intraluminal surfaces of the product are lined with mesothelial cells.

19. A method comprising the steps of:
shaping a mammalian mediastinal pleura tissue so that the mammalian mediastinal pleura tissue fits around at least a portion of a frame;
positioning the mammalian mediastinal pleura tissue around a mount;
positioning at least a part of the frame around the mammalian mediastinal pleura tissue positioned around the mount; and
connecting the mammalian mediastinal pleura tissue to the frame to form a tissue product.

20. The method of claim 19, wherein the tissue product is configured for mammalian treatment or therapy.
